(19) United States
(12) Patent Application Publication
(10) Pub. No.: US 2024/0095749 A1
(43) Pub. Date: Mar. 21, 2024

(54) Management of Real-Time Support From Internal and External Support Personnel

(71) Applicant: JustAskEvie, LLC, Leawood, KS (US)

(72) Inventor: Jean Dontee Tyler, Leawood, KS (US)

(21) Appl. No.: 18/371,361

(22) Filed: Sep. 21, 2023

Related U.S. Application Data
(60) Provisional application No. 63/408,592, filed on Sep. 21, 2022.

(51) Int. Cl. G06Q 30/015; G16H 10/60

(52) U.S. Cl. CPC G06Q 30/015; G16H 10/60

(57) ABSTRACT

An apparatus for assisting users with help requests. The apparatus includes a communications interface configured to be connected to a network; memory storing instructions; and a processor communicatively coupled to the communications interface and the memory. The processor can execute the instructions to receive, over the network, a help request from a requesting user and identify a set of support personnel for responding to the help request using selection criteria based, at least in part, on a similarity in roles of the requesting user and the set of support personnel. The processor can also execute the instructions to transmit a help request notification to the set of support personnel; receive an acceptance from a responding user from the set of support personnel; and establish a communication session between the requesting user and the responding user. Data transmitted over the communication session is used to satisfy the help request.

### CROSS-REFERENCE TO RELATED APPLICATIONS

This application claims priority to U.S. Provisional Application No. 63/408,592, filed on Sep. 21, 2022, entitled “MANAGEMENT OF REAL-TIME SUPPORT FROM INTERNAL AND EXTERNAL SUPPORT PERSONNEL” and is hereby incorporated herein by reference in its entirety as an example.

### BACKGROUND

Novel aspects of the present disclosure relate to the field of electronic medical records systems and more particularly to a system and method of managing support for users of EMR software systems, the support provided by internal support and external support personnel.

### BACKGROUND

Electronic medical records (EMRs) are digitized patient files that include medical histories, immunization records, diagnosis, allergies, lab results, medications, physician notes, etc. EMRs are made available to medical care service providers, such as doctors, nurses, and administrative staff via EMR software systems acquired by medical care facilities, such as hospital systems or clinics.

Currently, users of EMR software systems with questions are directed to a helpdesk, often offered by the EMR system vendor. Alternatively, some EMR software system users are instructed to log a ticket through email. In either of these conventional solutions, the EMR software system user can be required to wait until the proper support personnel can be identified and scheduled to provide the necessary assistance. This delay interrupts workflow for the medical care service provider and can be costly.

### SUMMARY OF THE INVENTION

Novel aspects of the present disclosure are directed to a web-based software platform that connects a network of EMR users to a network of support personnel in real-time through text messaging and push notifications. EMR users are identified by their role, mobile phone number and organization. Support personnel are identified by the roles they can support, organization, mobile phone number, and whether they are internally employed or externally employed. Organizations have criteria of coverage time. With these criteria, help requests are instantly triaged from the EMR user to the appropriate support personnel who matches. Details from the help request are collected and used for data analytics providing insight on most common questions asked, response time, session length and session frequency. This data is interpreted and used to provide interactive training.

In one aspect, an apparatus is disclosed which includes a communications interface configured to be connected to a network, memory storing instructions, and a processor communicatively coupled to the communications interface and the memory. The processor is configured to execute the instructions to: receive, over the network, a help request from a requesting user; identify a set of support personnel for responding to the help request using selection criteria based, at least in part, on a similarity in roles of the requesting user and the set of support personnel; transmit, over the network, a help request notification to the set of support personnel; receive, over the network, an acceptance from a responding user from the set of support personnel; and establish a communication session between the requesting user and the responding user. Data transmitted over the communication session is used to satisfy the help request.

In another aspect, an apparatus is disclosed which includes a communications interface configured to be connected to a network; memory storing instructions; and a processor communicatively coupled to the communications interface and the memory. The processor is configured to execute the instructions to receive user input to generate a help request from a requesting user and transmit the help requested over a network and to a remote computing device. The remote computing device is configured to: identify a set of support personnel for responding to the help request using selection criteria based, at least in part, on a similarity in roles of the requesting user and the set of support personnel, transmit, over the network, a help request notification to the set of support personnel, receive, over the network, an acceptance from a responding user from the set of support personnel; and establish a communication session between the requesting user and the responding user. The apparatus is also configured to participate in the communication session with the responding user. Data transmitted over the communication session is used to satisfy the help request.

Other aspects, embodiments and features of the invention will become apparent from the following detailed description of the invention when considered in conjunction with the accompanying figures. In the figures, each identical, or substantially similar component that is illustrated in various figures is represented by a single numeral or notation. For purposes of clarity, not every component is labeled in every figure. Nor is every component of each embodiment of the invention shown where illustration is not necessary to allow those of ordinary skill in the art to understand the invention.

### BRIEF DESCRIPTION OF THE FIGURES

The novel features believed characteristic of the invention are set forth in the appended claims. The invention itself, however, as well as a preferred mode of use, further objectives and advantages thereof, will be best understood by reference to the following detailed description of illustrative embodiments when read in conjunction with the accompanying figures, wherein:

FIG. 1 is a block diagram of a system for supporting EMR users according to an illustrative embodiment;

FIG. 2 is a block diagram of a communications device for use in supporting EMR users according to an illustrative embodiment;

FIG. 3 is a block diagram of a computing device for managing support of EMR users according to an illustrative embodiment;

FIG. 4 depicts an EMR system interface according to an illustrative embodiment;

FIG. 5 depicts a screen displaying a notification provided to support personnel according to an illustrative embodiment;

FIG. 6 depicts a screen displaying a help request queue provided to support personnel according to an illustrative embodiment;

FIG. 7 depicts a confirmation screen provided to a requesting user according to an illustrative embodiment;

FIG. 8 depicts a support summary screen provided to support personnel according to an illustrative embodiment;

FIG. 9 depicts a screenshare capability provided during a communication session between a medical care service provider and support personnel according to an illustrative embodiment;

FIG. 10 is a flowchart of a method for providing support to a user of an EMR system according to an illustrative embodiment;

FIG. 11 is a flowchart of another method for providing support to a user of an EMR system according to an illustrative embodiment;

FIG. 12 is a flowchart of yet another method for providing support to a user of an EMR system according to an illustrative embodiment; and

FIG. 13 is a flowchart of optional steps for implementing artificial intelligence in a method for providing support to a user of an EMR system according to an illustrative embodiment.

### DETAILED DESCRIPTION

A common trend in the medical field is the transition from paper medical records to electronic medical records, which forces new medical care service providers to learn new EMR software systems. Even medical care service providers experienced with EMR software systems may still be required to learn new EMR software systems over time, i.e., when changing employment, when employers adopt new EMR software systems, when medical groups expand coverage to medical care facilities implementing different EMR software systems, or when EMR software systems undergo large-scale updates. Thus, the large number of EMR software systems, the large number of users of EMR software systems, and the frequency of events requiring users of EMR software systems to learn (or relearn) how to use EMR software systems necessitates the existence of support personnel that can provide timely and cost-effective responses to help requests.

Novel aspects of the present disclosure recognize the need for a timely and cost-effective solution to provide support to users of EMR software systems. To this end, the novel aspects of this disclosure provide a way for users of an EMR software system, i.e., medical care service providers, to solicit help from support personnel that can include internal support personnel employed at the same organization, e.g., same medical care facility. The internal support personnel can be co-workers having the same or similar role(s), e.g., peer-level employees within the same organization, as well as external support personnel employed by a third party, i.e., an organization that does not employ the user soliciting help. Examples of the external support personnel can include employees of the EMR software vendor and/or peer-level employees working at other organizations with the same or similar role(s). For example, the external support personnel can also be a medical care service provider employed at a different medical care facility from the user of the EMR software system generating the original help request, but with requisite expertise with operating the same EMR software system. The novel aspects of this disclosure can reduce the reliance on expensive helpdesk solutions that are disfavored because they are oftentimes paid to be on call in event that assistance may eventually be required, they are unable to quickly adjust to higher volume cycles (e.g., onboarding of large number of new medical care service providers, expanded coverage to a medical care facility, etc.), they may not be knowledgeable about the customized EMR software system or the protocols implemented at a particular medical care facility, among other reasons.

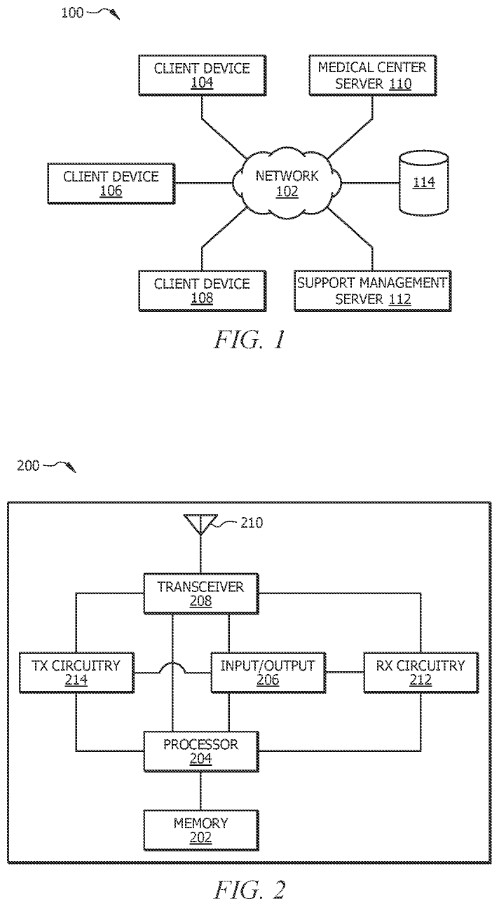

FIG. 1 is a block diagram of a system for providing support to EMR users according to an illustrative embodiment. Generally, the system 100 includes a network 102 that facilitates communication among a plurality of client devices 104, 106, and 108 to allow one or more users, i.e., medical care service providers (not shown), to communicate with support personnel using conventional communication protocols such as text messaging, push notifications, screen share, and phone calls.

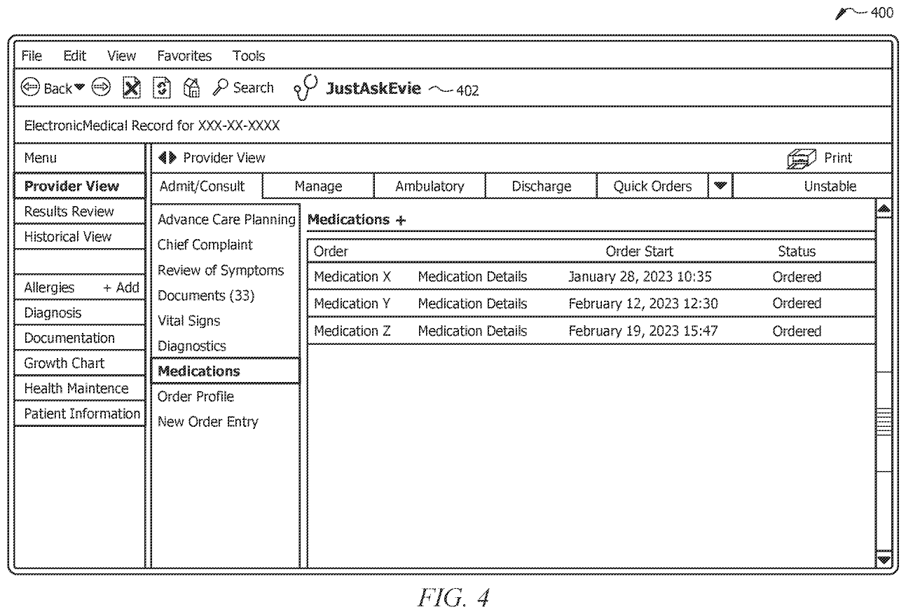

In an exemplary use case, the system 100 allows a medical care service provider to operate a client device 104 to access an EMR software system, which can be hosted on a medical center server 110 or a server hosted by the EMR software system vendor (not shown). Medical records maintained by the EMR software system can be stored locally to the medical center server 110 or remotely, such as in network accessible storage 114. Help requests can be generated by the medical care service provider via the EMR system interface by clicking a UI element, such as a button integrated into the EMR system interface, as can be seen in FIG. 4. Upon clicking the button, a support management application, which can be hosted by the support management server 112 in some embodiments, is launched to process the help request and select one or more support personnel to provide the requested assistance.

The support management application can select the one or more support personnel based on selection criteria. Non-limiting examples of the selection criteria can include the time that the help request was received, the role of the support personnel (e.g., whether the support personnel is a physician, nurse, medical technician, etc.), a place of employment of the support personnel, the specialty of the of the support personnel (e.g., whether both are oncologists, dermatologists, etc.), a number of years of experience, existence of training certificates, feedback ratings from other users, and speed at resolving issues, among others. The selection criteria can be stored in each user's user profile, which can be stored locally to the medical center server 110 or remotely, such as in network accessible storage 114.

The following non-limiting example illustrates the implementation of the selection criteria. During normal operating hours of a medical care facility, support personnel can be selected from a first group of support personnel that are also working at the same medical care facility as the user generating the help request, i.e., internal support personnel. After normal operating hours, e.g., on the night shift, the support personnel can be selected from a second group of support personnel working as an external support team. In some instances, there may be some time period where both groups of support personnel are available to provide support. Examples of this overlapping time period includes shift changes, lunch hours, holidays, after new feature releases, or during periods of abnormal help request volume. For example, if there are an insufficient number of members in a first group of support personnel available to address all the help requests, then help from members of a second group of support personnel may be requested.

At least some of the novel aspects of this disclosure recognizes that experienced users of an EMR system within the same organization as a user requesting help (i.e., internal support personnel) are best suited for answering questions about use of that EMR system at that organization. Experienced users of that EMR system at another similar organization (i.e., external support personnel), but who have the same or similar roles as the user generating the help request, likely have relevant insight in the operation of that EMR system and are the next best suited for answering questions about the use of that EMR system. Thus, a helpdesk-like solution that sends usage questions to a first group of peer-level users of the EMR system at the same organization, and then to a second group of peer-level users of the EMR system at a different organization, can provide the most relevant and cost-effective expertise for addressing usage questions.

In another example, a request for assistance that is not addressed within a threshold amount of time could result in conscription of additional help. For example, if a help request is not addressed by a member from a first group of internal support personnel within a threshold amount of time, i.e., 30 seconds, then the request may be sent to a member of a second group of support personnel, e.g., external support personnel. In an alternate example, if the external support personnel is scheduled to provide coverage for answering help requests, but a member of the external support personnel is unable to respond to a help request within the threshold amount of time, then the help request can be redirected to one or more members of the internal support personnel. In each of these examples, the members of each of the different groups of support personnel can be identified and/or selected for responding to a help request based on the previously mentioned criteria, such as role. Thus, a help request generated by an oncologist may be forwarded to a medical care service provider that is at least a physician, but in another embodiment the help request may be forwarded to another oncologist initially and then redirected to another physician if no oncologists are available.

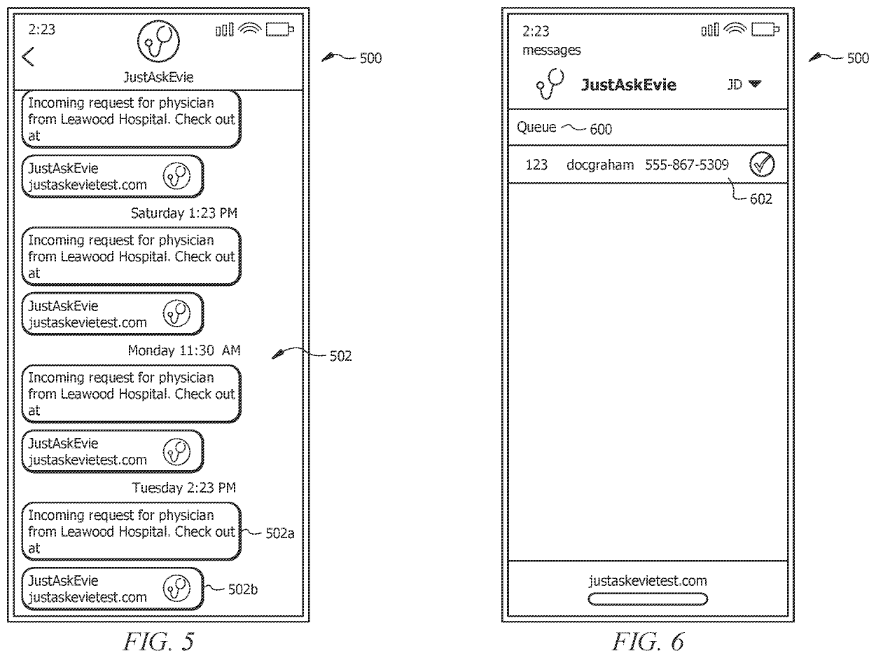

The selected support personnel can be sent a help request notification indicating that their assistance is requested. In a non-limiting embodiment, the help request notification is a push notification transmitted over text message to the support personnel's mobile communications device. An example of the push notification is shown in FIG. 5. The notification can include a link to a request queue that displays outstanding help requests. An example of the request queue is shown in FIG. 6.

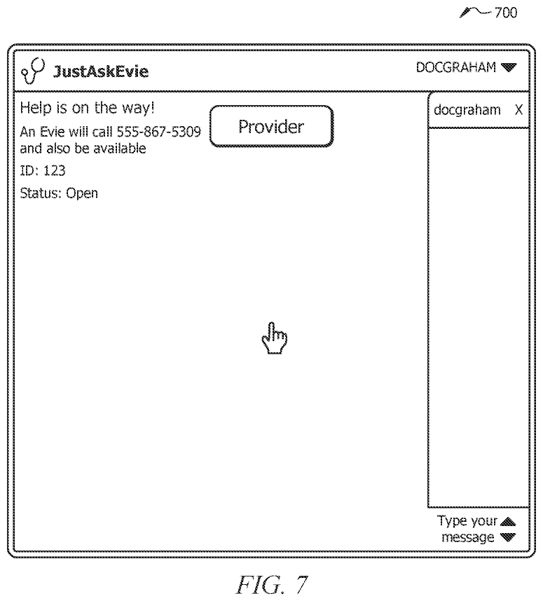

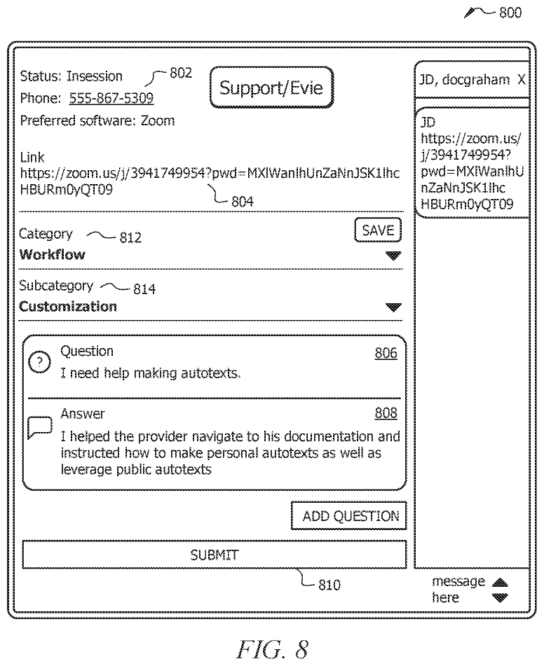

The support personnel selecting an entry on the request queue can trigger a confirmation message to be sent by the support management application, as can be seen in FIG. 7. The support personnel can then be directed to a support summary screen, shown in FIG. 8, which provides contact information for establishing a communication session between the medical care service provider and the support personnel. During the communication session the medical care service provider requesting assistance can ask questions that can be answered by the support personnel. The screen share capability facilitates the exchange of information usable to resolve the help request. After the issue has been resolved, the support personnel can provide additional feedback to identify the issue at hand and the solution(s) provided, which can be used to improve the EMR software system or improve training on the usage of the EMR software system.

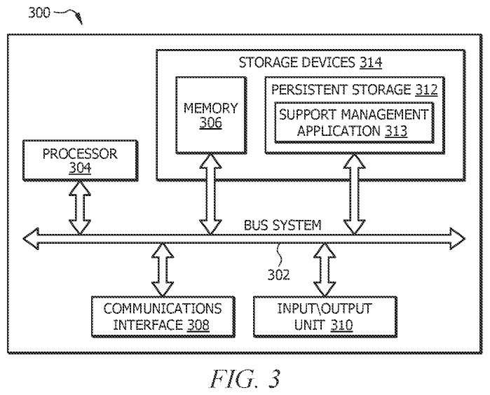

The support management application, which is shown support management application 313 hosted on server 300 in FIG. 3, can be a comprehensive, cloud-based software solution that can be tailored to help manage help requests generated by users of a EMR software system. The cloud-based software solution can be accessible via a web browser executing on a client device, such as client device 104. In another embodiment, the support management application can be a downloadable application executing locally on a client device, but using data stored remotely from the client device, e.g., on network accessed storage 114 or medical center server 110. In yet another embodiment, the support management application can be distributed over two or more computing devices, such as a client device and a server, on two or more client devices, or on two or more servers.

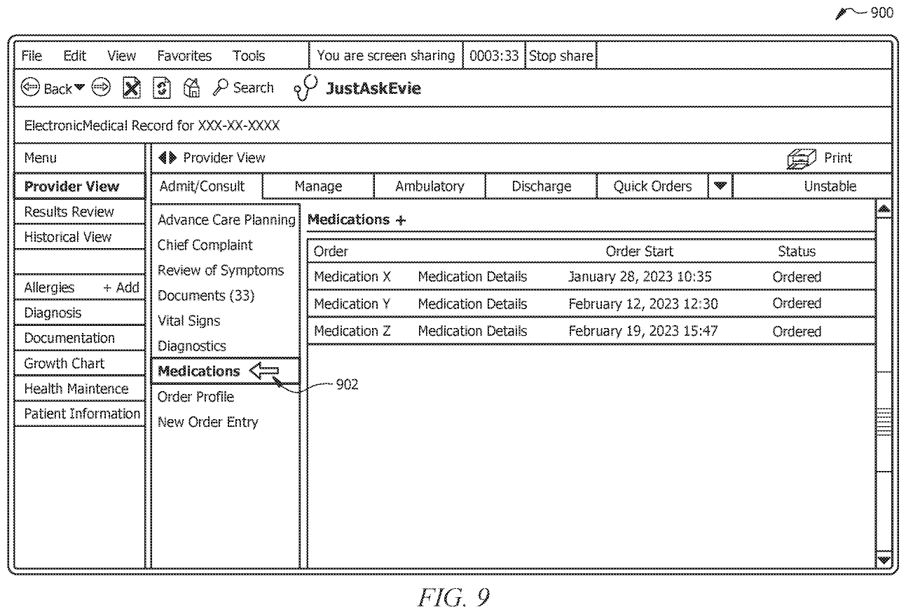

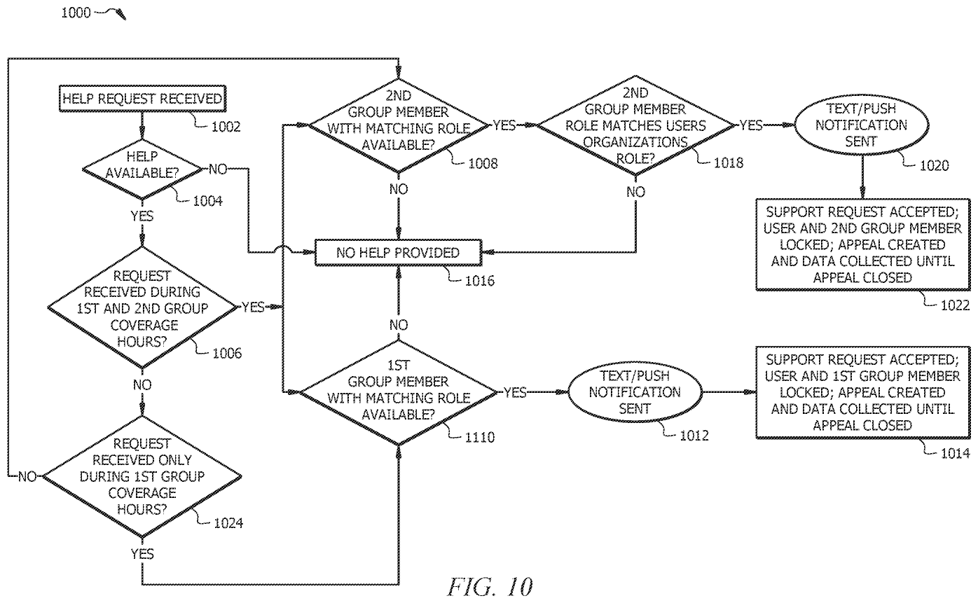

The support management application 313 can interface with native communications apps on the client devices to allow for a bi-directional flow of data during a communication session. For example, the support management application 313 can be used to launch a telephone call or a videoconference with screenshare capability using any number of conventional videoconferencing applications. In instances where the communication session includes screen sharing, the support management application can also provide a pointer controllable by the support personnel that can be used to direct the medical care service provider's attention to various parts of the shared screen, as described in more detail in FIG. 9 that follows.

Examples of client devices 104, 106, and 108 can include cell phones, tablets, desktop computers, or any other form of communications device, which permit communication via conventional means, such as the exchange of emails, text messages, phone calls, and/or videoconference calls. An example of a client device is shown in more detail in FIG. 2 that follows.

The network 102 can include the internet, the Public Switched Telephone Network (PSTN), cellular networks, and local area networks, among others. Communication over the network 102 can be achieved using various forms of communications equipment and protocols. While client devices 104, 106, and 108 are depicted as communicating through communications links via network 102, in other embodiments, the client devices 104, 106, and 108 can communicate via device-to-device communications protocols. Based on the communications sessions conducted on one or more of the client devices 104, 106, and 108, a medical care service provider can generate help requests that are answered by support personnel formed a first group of internal support personnel, a second group of external support personnel, or a combination of the two groups.

FIG. 2 is a block diagram of a communications device for use in supporting EMR users according to an illustrative embodiment. The client device 200 is provided for illustration only. The client devices 104, 106, and 108 in FIG. 1 can have the same or similar configuration as the client device 200 in FIG. 2.

Client device 200 includes memory 202 storing instructions that can be executed by processor 204 for controlling the operation of the client device 200. For example, the memory can store an operating system and one or more applications that can be executed by the processor 204. The memory 202 can include random access memory (RAM), Flash memory, and/or read-only memory (ROM).

I/O 206 is one or more input/output (I/O) devices of the client device 200. Examples of I/O devices include, but are not limited to, a microphone, a speaker, a camera, a touch screen, a keypad. I/O 206 enables a user to interact with the client device 200 to communicate with a user of another client device, i.e., via a phone call, text message, email, or videoconference, for receiving or providing support according to the novel aspects disclosed in this disclosure. In some embodiments, I/O 206 also includes I/O interfaces that provide the client device 200 with communications paths with other devices, such as other client devices and peripherals.

The transceiver 208 provides a wireless communications capability with a network, such as network 102 in FIG. 1. Incoming signals are received by the transceiver 208 from the antenna 210 and processed by the receive (RX) circuitry 212, which processes the signal and transmits the processed signal to an I/O device, such as a speaker, if the processed signal is for voice data. The processed signal can also be transmitted to the processor 204 for further processing before presentation to a user on another I/O device, such as a screen, if the processed signal is for other forms of data, such as web browsing data. Outgoing signals transmitted by the transceiver 208 from the antenna 210 are received from transmit (TX) circuitry 214. The TX circuitry 214 can receive voice data from a microphone, or other forms of outgoing data, such as web data, e-mail, or application data, from the processor 204.

The client device 200 in FIG. 2 is depicted as a mobile phone, the client device 200 can be any other conventional client computing devices such as tablets, laptop computers, and desktop computers. For example, the transceiver depicted in the client device 200 can be replaced by a network communications interface that can support wired or wireless communication over a user's home network.

A user of client device 200 can operate EMR system software and, if necessary, request help from support personnel formed from a first group of internal support personnel and/or a second group of external support personnel. The help can be facilitated by a support management application hosted locally to the client device 200 or remotely from the client device 200, such as in a medical care facility server accessible via the network 102. Another user of a different client device 200 can provide the requested help via the support management application once notified of the help request. Help can be provided using any one or more conventional communications protocols, such as voice communication, videoconferencing communication, and screensharing.

FIG. 3 is a block diagram of a computing device for managing support of EMR users according to an illustrative embodiment. For example, the server 300 can be a support management server 112 in FIG. 1.

Server 300 includes a bus system 302 that supports communication between at least one processor 304, at least one storage device 314, at least one communications interface 308, and at least one input/output (I/O) unit 310.

The memory 306 and a persistent storage 312 are examples of storage devices 314, which represent any structure(s) capable of storing and facilitating retrieval of information (such as data, program code, and/or other suitable information on a temporary or permanent basis). The memory 306 may represent a random-access memory or any other suitable volatile or non-volatile storage device(s). The persistent storage 312 may contain one or more components or devices supporting longer-term storage of data, such as a read only memory, hard drive, Flash memory, or optical disc.

The processor 304 may execute instructions that may be loaded into the memory 306. The processor 304 may include any suitable number(s) and type(s) of processors or other devices in any suitable arrangement. Example types of processors 304 include microprocessors, microcontrollers, digital signal processors, field programmable gate arrays, application specific integrated circuits, and discreet circuitry.

The communications interface 308 may support communications with other systems or devices. For example, the communications interface 308 could include a network interface card or a wireless transceiver facilitating communications over the network 102. The communications interface 308 may support communications through any suitable physical or wireless communication link(s).

The I/O unit 310 may allow for input and output of data. For example, the I/O unit 310 may provide a connection for user input through a keyboard, mouse, keypad, touchscreen, or other suitable input device. The I/O unit 310 may also send output to a display, printer, or other suitable output device.

As described in more detail below, the server 300 can be implemented as a support management server in a networked computing system that can host a support management application 313 configured to process help requests received from a medical care service provider seeking assistance on EMR system usage, select one or more support personnel that can include members of a first group of internal support personnel and/or members of a second group of external support personnel, facilitate a communication session where the requisite assistance is provided, and collect and analyze data related to the help request.

FIG. 4 depicts an EMR system interface according to an illustrative embodiment. The EMR system interface 400 can be displayed to a user operating computing device, such as client devices 104, 106, and 108 in FIG. 1, or computing device 200 in FIG. 2. More specifically, the EMR system interface 400 can be displayed on a web browser presented to a user viewing an I/O device, such as a screen or monitor. In this exemplary embodiment, the EMR system interface 400 includes a user interface (UI) element 402, which is in the form of a button that can be pressed by an I/O device such as a mouse or touchscreen. A medical care service provider using the EMR system interface 400 can press the UI element 402 if the medical care service provider requires assistance. Pressing the UI element 402 generates a help request and launches a support management application 313.

FIG. 5 depicts a screen displaying a notification provided to support personnel according to an illustrative embodiment. The screen 500 is an I/O device of a computing device, client devices 104, 106, and 108 in FIG. 1, or computing device 200 in FIG. 2. Support personnel, such as members of a first group of internal support personnel or members of a second group of external support personnel, operating a client device can receive a help request notification 502 indicating that a help request has been received. In the example in FIG. 5, the help request notification 502 includes a text message 502a indicating the role of the medical care service provider requesting assistance, i.e., physician, and the medical care facility employing the medical care service provider, i.e., Leawood Hospital. The information provided in the help request notification 502 can be derived from the biographical data included in user profile established for each user of the EMR system. The notification 502 can also include selectable link 502b that directs the support personnel to a help request queue, which is shown in FIG. 6 that follows.

FIG. 6 depicts a screen displaying a help request queue provided to support personnel according to an illustrative embodiment. The help request queue 600 displayed on screen 500 lists outstanding help requests. In this example, only one help request entry 602 is displayed. The help request entry 602 identifies the requesting user as well as some form of contact information. In this example, the contact information is a telephone number. Selection of the help request entry 602 by support personnel can cause the support management application 313 to display a confirmation screen the medical care service provider requesting help, as shown in FIG. 7, and then display a support summary screen 800 to the support personnel, as shown in FIG. 8.

FIG. 7 is a confirmation screen provided to a requesting user according to an illustrative embodiment. The confirmation screen 700 can be presented to the medical care service provider requesting help via the support management application 313. The confirmation screen 700 can be presented on the client device operated by the medical care service provider which generated the help request.

FIG. 8 displays a support summary screen provided to support personnel according to an illustrative embodiment. The support summary screen 800 can be provided to the support personnel, which provides contact information, such as a telephone number 802 and a hyperlink 804 that can be used to launch a screen sharing session. In some embodiments, the support personnel can call the telephone number 802 to establish voice communications with the medical care service provider requesting help. Then the support personnel can select the hyperlink 804 to initiate a screen sharing session where the support personnel can view the EMR system interface and provide the requisite assistance. An example of the screen sharing session is shown in FIG. 9 that follows.

During the communications session or sometime thereafter, the support personnel can provide some data describing the type of issue addressed during the support session in text field 806 and also the solution that was presented in text field 808. The collected data can be analyzed and used for improving the EMR software system or for improving training sessions to support use of the EMR software system. In addition, the support personnel can populate field 812 with a category of the issued addressed field 814 with a subcategory of the issued addressed. The category and/or subcategory can be inputted by a keyboard or selected from a dropdown list by selecting the dropdown menu icon. Selecting the SUBMIT UI element 810 can end the communication session and close the support summary screen 800.

FIG. 9 depicts a screenshare capability provided during a communication session between a medical care service provider and support personnel according to an illustrative embodiment. The shared screen 900 displays the EMR system interface 400 originally presented to the medical care service provider. The shared screen 900 can allow the medical care service provider to better describe the issue underlying the help request. In some embodiments, the software management application 313 allows the support personnel to control a pointer 902 that allows that support personnel to direct the medical care service provider's attention to various parts of the EMR system interface 400.

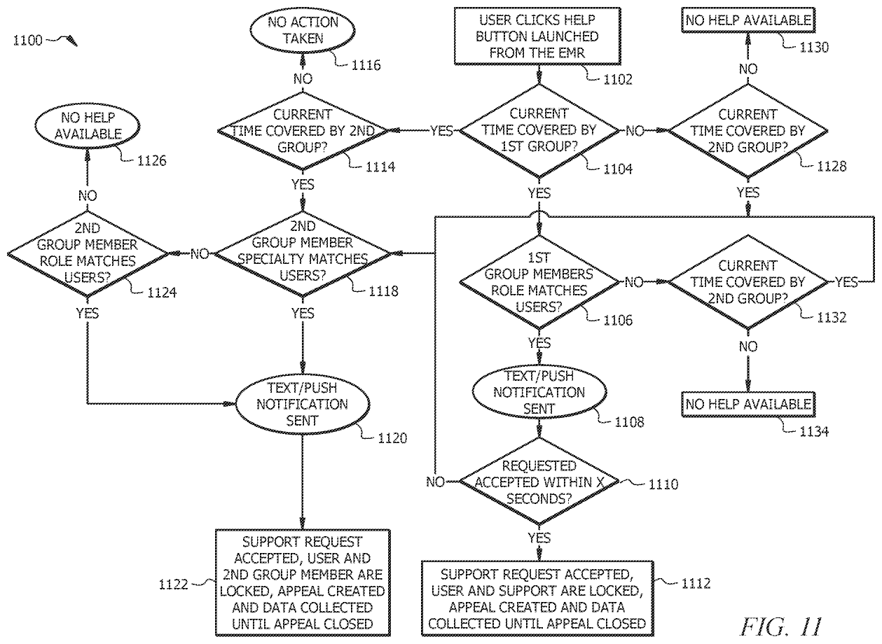

FIG. 10 is a flowchart of another method for providing support to a user of an EMR system according to an illustrative embodiment. Steps of flowchart 1000 can be implemented in a support management server, such as support management server 112 in FIG. 1.

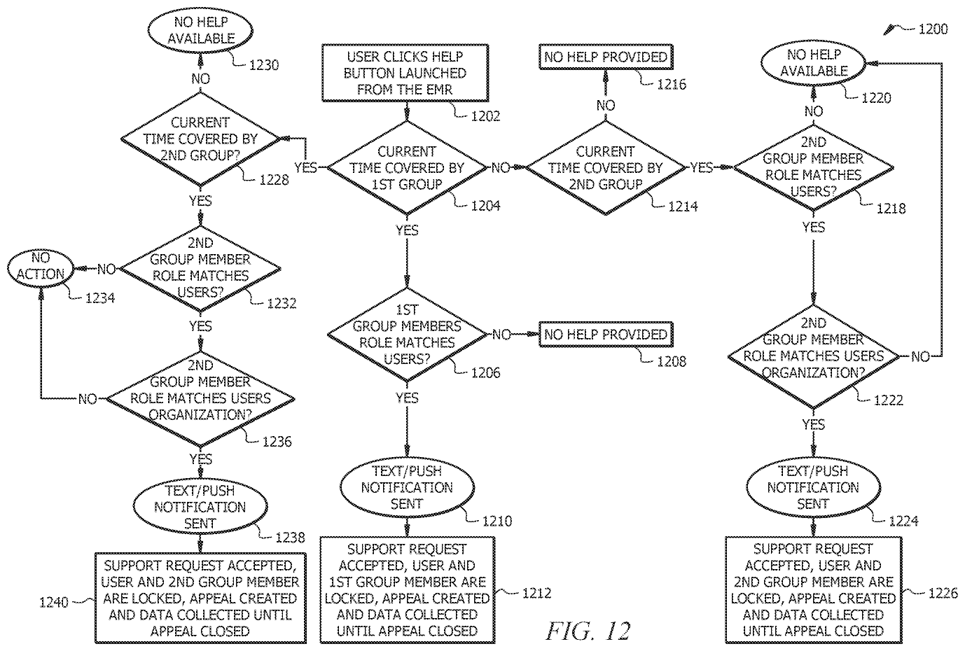

Flowchart 1000 begins at step 1002 by receiving a help request. The help request can be generated by a user of an EMR system. The help request can be generated by pressing a button, like UI element 402 in FIG. 4. A determination is made in step 1004 as to whether help is available. If help is not available, then flowchart 1000 proceeds to step 1016 and no help is provided. If help is available, then flowchart 1000 proceeds from step 1004 to step 1006 when a determination is made as to whether the help request is received during coverage by a first group of support personnel and a second group of support personnel. In this non-limiting example and the examples described in FIGS. 11 and 12, the first group of support personnel is formed from peer-level co-workers (i.e., internal support personnel) and the second group of support personnel is formed from peer-level employees employed by a different organization, but who all have experience using the EMR system at issue (i.e., external support personnel). If the request is made during first group coverage and second group coverage, then flowchart 1000 proceeds to steps 1008 and 1010. The flowchart 1000 can proceed to steps 1008 and 1010 concurrently or sequentially.

In step 1008, a determination is made as to whether any member of the second group with a role matching the requesting user's role is available. In step 1010, a determination is made as to whether any member of the first group with a role matching the requesting user's role is available. If a member of the first group with the role matching the requesting user's role is not available, then flowchart 1000 proceeds from step 1010 to step 1016 and no help is provided. If a member of the first group with the role matching the requesting user's role is available, then a help request notification is provided in step 1012 to the first group member with the role matching the requesting user's role. The notification can be in the form of a text message or other form of push notification. The notification provides the requisite information to allow a communication session to be established. Thereafter, a communication session is established in step 1014 between the requesting user and the member of the first group upon acceptance of the help request. Data can be collected during the communication session which can be used to identify the issue encountered and the solution presented.

Returning back to step 1008, if the determination is made that a member of the second group with a role matching the requesting user's role is not available, then flowchart 1000 proceeds to step 1016. However, if a member of the second group with a role matching the requesting user's role is available, then a subsequent determination is made in step 1018 as to whether a member of the second group with a role that matches the user's organization's role is available. If a member of the second group with a role that matches the user's organization's role is not available, then flowchart 1000 proceeds to step 1016 and no help is provided. However, if a member of the second group with a role that matches the user's organization's role is available, the flowchart 1000 proceeds to step 1020 and a help request notification is sent to the member of the second group whose role matches the user's organization's role. The help request notification can be in the form of a text message or other form of push notification. The help request notification provides the requisite information to allow a communication session to be established. Thereafter, a communication session is established in step 1020 between the requesting user and the member of the second group upon acceptance of the help request. Data can be collected during the communication session which can be used to identify the issue encountered and the solution presented.

Returning to step 1006, if a determination is made that the request is not received during first group and second group coverage hours, then flowchart 1000 proceeds to step 1024 where a subsequent determination is made as to whether the request is received only during first group coverage hours. If the request is not received only during first group coverage hours, then the request is received during second group coverage hours and flowchart 1000 returns to step 1008. However, if the request is received only during first group coverage hours, then the flowchart 1000 returns to step 1010.

FIG. 11 is a flowchart of still another method for providing support to a user of an EMR system according to an illustrative embodiment. Steps of the flowchart 1100 can be implemented in a support management server, such as support management server 112 in FIG. 1.

Flowchart 1100 begins at step 1102 by receiving a help request. The help request can be triggered by a requesting user interacting with a UI element presented in the EMR system, as previously described in FIG. 4. In Step 1104 determination is made as to whether coverage by a first group is available. In a non-limiting embodiment, the determination is made based on whether the help request was made during coverage hours assigned to the first group. If first group coverage is available, then flowchart 1100 can proceed to step 1106 where a subsequent determination is made as to whether any member of the first group matches the requisite selection criteria. In a non-limiting embodiment, the selection criteria can include a similarity in the first group member(s)' role to that of the requesting user's role. If the selection criteria is met, then a help request notification is sent to the member(s) of the first group in step 1108.

In step 1110, a determination is made as to whether the help request has been accepted within a threshold amount of time. If the request has been accepted within the threshold amount of time, then a communication session is established between the requesting user and the member of the first group in step 1112. Data can be collected during the communication session which can be used to identify the issue encountered and the solution presented.

In some embodiments, coverage by the first group and the second group can overlap, which can provide increased responsiveness to address help requests, or to provide additional coverage in times of need, i.e., during shift changes, during onboarding of a large number of new medical care service providers, during EMR system updates, etc. In these embodiments, flowchart 1100 can optionally proceed from step 1104 to 1106 and also to step 1114, where a determination is made as to whether coverage by the second group is available. The flowchart 1100 can proceed from step 1104 to step 1106 and concurrently to step 1114 or sequentially. In these embodiments, the determination made in step 1114 can be based on a time that the help request was received from the requesting user, i.e., whether the help request was received during coverage hours of the second group. If the request is received when coverage by the second group is not available, then flowchart 1100 proceeds from step 1114 to step 1116 and no action is taken. If coverage by the second group is available, the flowchart 1100 proceeds from step 1114 to step 1118 where a determination is made as to whether a member of the second group is available with a specialty that matches the requesting user's specialty. If a member of the second group is available with the specialty that matches the requesting user's specialty, then flowchart 1100 proceeds to step 1120 and a help request notification is sent to the member of the second group that matches the requesting user's specialty. The help request notification provides the requisite information to allow a communication session to be established. In step 1122, a communication session is established between the requesting user and the member of the second group in response to the member of the second group accepting the help request. Data can be collected during the communication session which can be used to identify the issue encountered and the solution presented.

Returning to step 1118, if a determination is made that a specialty of a member of the second group does not match the requesting user's specialty, then flowchart 1100 proceeds to step 1124 where a determination is made as to whether a role of the member of the second group matches the requesting user's role. If the role of the member of the second group does not match the requesting user's role, then flowchart 1100 continues to step 1126 and the requesting user is provided with a message indicating that no help is available. But if the role of the member of the second group does match the requesting user's role, as determined in step 1124, then flowchart 1100 continues to step 1120.

Returning again to step 1104, if the determination is made that coverage by a first group is not available, then flowchart 1100 proceeds to step 1128 where a determination is made as to whether coverage by a second group is available. If coverage by the second group is not available, then the requesting user is provided with a message indicating that no help is available in step 1130. If coverage by the second group is available, then flowchart 1100 proceeds to step 1118.

Returning to step 1106, if the determination is made that the selection criteria is not met, e.g., the support team member(s)′ role does not match the requesting user's role, then flowchart 1100 proceeds to step 1132 where a determination is made as to whether coverage by a second group is available. If coverage by the second group is not available, then the requesting user is provided with a message indicating that no help is available in step 1134. If coverage by the second group is available, then flowchart 1100 proceeds to step 1118.

Returning back to step 1110, if the determination is made that the help request has not been accepted within a threshold amount of time, then flowchart 1100 proceeds to step 1118.

FIG. 12 is a flowchart of yet another method for providing support to a user of an EMR system according to an illustrative embodiment. Steps of the flowchart 1200 can be implemented in a support management server, such as support management server 112 in FIG. 1.

Flowchart 1200 begins at step 1202 by receiving a help request. The help request can be triggered by a requesting user interacting with a UI element presented in the EMR system, as previously described in FIG. 4. In Step 1204 determination is made as to whether coverage by the first group is available. In a non-limiting embodiment, the determination is made based on whether the help request was made during the coverage hours assigned to the first group. If coverage by the first group is available, then flowchart 1200 can proceed to step 1206 where a subsequent determination is made as to whether any member of the first group matches the requisite selection criteria. In a non-limiting embodiment, the selection criteria can include a similarity in the first group member(s)′ role to that of the requesting user's role. If the selection criteria is not met, then flowchart 1200 proceeds to step 1208 and the requesting user is provided with a message indicating that no help is available. However, if the selection criteria is met in step 1206, then a help request notification is sent to the member(s) of the first group in step 1210. The help request notification provides the requisite information to allow a communication session to be established. Then, in step 1212, a communication session is established between the requesting user and the member of the first group in step 1212. Data can be collected during the communication session which can be used to identify the issue encountered and the solution presented.

Returning to step 1204, if the determination is made that coverage by the first group is not available, then flowchart 1200 proceeds to step 1214 where a determination is made as to whether coverage by the second group is available. The determination made in step 1214 can be based on a time that the help request was received from the requesting user, i.e., whether the help request was received during coverage hours assigned to the second group. If coverage by the second group is not available, then flowchart 1200 proceeds to step 1216 and the requesting user is provided with a message indicating that no help is available. However, if coverage by the second group is available, then a determination can be made in step 1218 as to whether a member of the second group with a role that matches the requesting user's role is available. If no member of the second group is available with a role that matches the requesting user's role, then flowchart 1200 proceeds to step 1220 and no help is provided to the requesting user. If the determination is made that a member of the second group is available with a role that matches the requesting user's rule then a determination is made in step 1222 as to whether the role of the member of the second group matches the requesting user's organization's role.

If no member of the second group has a role that matches the requesting user's organization's role in step 1222, then flowchart 1200 returns to step 1220 and no help is provided. If a member of the second group has a role that matches the requesting user's organization's role and is available, then a help request notification is sent to the member of the second group in step 1224. The help request notification provides the requisite information to allow a communication session to be established. Then, a communication session is established in step 1226 between the requesting user and the member of the second group having the requisite role upon acceptance of the help request. Data can be collected during the communication session which can be used to identify the issue encountered and the solution presented.

In some embodiments, coverage by the second group and the first group can overlap, which can provide increased responsiveness to address help requests, or to provide additional coverage in times of need, i.e., during shift changes, during onboarding of a large number of new medical care service providers, during EMR system updates, etc. In these embodiments, flowchart 1200 can optionally proceed from step 1204 to 1206 and also to step 1228, where a determination is made as to whether coverage by the second group is available. The flowchart 1200 can proceed from step 1204 to step 1206 and concurrently to step 1228 or sequentially. In these embodiments, the determination made in step 1228 can be based on a time that the help request was received from the requesting user, i.e., whether the help request was received during coverage hours assigned to the second group. If the request is received when coverage by the second group is not available, then flowchart 1200 proceeds from step 1228 to step 1230 and support by the second group is not provided to the requesting user. If coverage by the second group is available, the flowchart 1200 proceeds from step 1228 to step 1232 where a determination is made as to whether a member of the second group is available with a role that matches the requesting user's rule.

If the determination is made in step 1232 that a member of the second group is not available that matches the requesting user's role, then flowchart 1200 proceeds to step 1234 and no action is taken. If a member of the second group is available with a role that matches the requesting user's role, then flowchart 1200 proceeds to step 1236 where a determination is made as to whether the member of the second group has a role that matches the requesting user's organization's role. If no member of the second group has a role that matches the requesting user's organization's role, then flowchart 1200 proceeds to step 1234 and no help is provided. If a member of the second group has a role that matches the requesting user's organization's role and is available, then a help request notification is sent to that member of the second group in step 1238. The help request notification provides the requisite information to allow a communication session to be established. Then, in step 1240, a communication session is established between the requesting user and the member of the second group upon acceptance of the help request. Data can be collected during the communication session which can be used to identify the issue encountered and the solution presented.

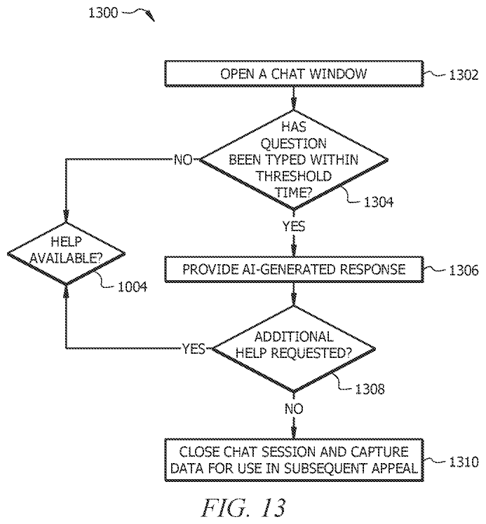

FIG. 13 is a flowchart of steps for implementing artificial intelligence in a method for providing support to a user of an EMR system according to an illustrative embodiment. The steps of flowchart 1300 allows AI to attempt to answer commonly asked questions before the questions are sent to a person. Certain steps of flowchart 1300 can be implemented as sub-steps of a step for receiving a help request, such as step 1002 in FIG. 10, step 1102 in FIG. 11, and step 1202 in FIG. 12. For example, when a help request is received in step 1002, flowchart 1300 can proceed to step 1302 by opening a chat window. The chat window can be presented to the user who generated the help request. The chat window can be presented to the user on a communications device being operated by the user, such as client device 104.

In step 1304, a determination can then be made as to whether a question has been typed within a predetermined amount of time. If a question has not been typed into the chat window within the predetermined amount of time, then the flowchart 1300 proceeds to make a determination as to whether help is available, e.g., by proceeding to step 1004 in flowchart 1000. However, if a question has been typed into the chat window within the predetermined amount of time, then the flowchart 1300 proceeds to step 1306 and provides an AI-generated response. The flowchart 1300 continues to step 1308 and asks the user if additional help is requested. If no additional help is requested, then flowchart 1300 proceeds to step 1310 where the chat session is closed and data from the chat session is captured as data for subsequent appeal. However, if additional help is requested, then flowchart 1300 can direct the requesting user to additional help, e.g., by proceeding to step 1004.

Although embodiments of the invention have been described with reference to several elements, any element described in the embodiments described herein are exemplary and can be omitted, substituted, added, combined, or rearranged as applicable to form new embodiments. A skilled person, upon reading the present specification, would recognize that such additional embodiments are effectively disclosed herein. For example, where this disclosure describes characteristics, structure, size, shape, arrangement, or composition for an element or process for making or using an element or combination of elements, the characteristics, structure, size, shape, arrangement, or composition can also be incorporated into any other element or combination of elements, or process for making or using an element or combination of elements described herein to provide additional embodiments.

Additionally, where an embodiment is described herein as comprising some element or group of elements, additional embodiments can consist essentially of or consist of the element or group of elements. Also, although the open-ended term “comprises” is generally used herein, additional embodiments can be formed by substituting the terms “consisting essentially of” or “consisting of.” As used herein, the term “set” means one or more. Thus, a set of helpers/support personnel can be a single person or two or more people.

While this invention has been particularly shown and described with reference to preferred embodiments, it will be understood by those skilled in the art that various changes in form and detail may be made therein without departing from the spirit and scope of the invention. The inventors expect skilled artisans to employ such variations as appropriate, and the inventors intend the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

We claim:

1. An apparatus comprising:
a communications interface configured to be connected to a network;
memory storing instructions;
a processor communicatively coupled to the communications interface and the memory, wherein the processor is configured to execute the instructions to:
receive, over the network, a help request from a requesting user;
identify a set of support personnel for responding to the help request, wherein the set of support personnel is selected using selection criteria based, at least in part, on a similarity in roles of the requesting user and the set of support personnel,
transmit, over the network, a help request notification to the set of support personnel;
receive, over the network, an acceptance from a responding user from the set of support personnel; and
establish a communication session between the requesting user and the responding user, wherein data transmitted over the communication session is used to satisfy the help request.

2. The apparatus of claim 1, wherein:
the help request is received from a user operating a software program on a first computing device; and
the acceptance is received from the responding user operating a second computing device located remotely from the first computing device.

3. The apparatus of claim 1, wherein:
the software program is electronic medical records (EMR) software; and
the help request is generated by interacting with a user-interface element integrated into a user-interface of the EMR software.

4. The apparatus of claim 1, wherein the communication session includes at least one of a phone call, videoconferencing call, or a screen sharing session.

5. The apparatus of claim 1, wherein the set of support personnel includes a first support team formed from members of an organization employing the requesting user.

6. The apparatus of claim 5, wherein the set of support personnel includes a second support team formed from members of an organization not employing the requesting user.

7. The apparatus of claim 6, wherein the selection criteria also includes a coverage time of the first support team and a coverage time of the second support team.

8. The apparatus of claim 1, wherein the help request notification is a push message.

9. The apparatus of claim 1, wherein the processor is further configured to execute the instructions to:
receive data indicating an issue addressed during the communication session; and
receive data indicating a solution provided to resolve the issue raised during the communication session.

10. The apparatus of claim 9, wherein the processor is further configured to execute the instructions to:
identify issues to include in future training sessions based on the data indicating the issue addressed during the communication session and the data indicating the solution to the issue addressed during the communication session.

11. An apparatus comprising:
a communications interface configured to be connected to a network;
memory storing instructions;
a processor communicatively coupled to the communications interface and the memory, wherein the processor is configured to execute the instructions to:
receive user input to generate a help request from a requesting user;
transmit the help requested over a network and to a remote computing device configured to:
identify a set of support personnel for responding to the help request using selection criteria based, at least in part, on a similarity in roles of the requesting user and the set of support personnel,
transmit, over the network, a help request notification to the set of support personnel, and
receive, over the network, an acceptance from a responding user from the set of support personnel; and
participate in a communication session with the responding user, wherein data transmitted over the communication session is used to satisfy the help request.

12. The apparatus of claim 11, wherein:
the help request is received by a first computing device located remotely from the apparatus; and
the acceptance is received from the responding user operating a second computing device located remotely from the apparatus and the first computing device.

13. The apparatus of claim 11, wherein:
the software program is electronic medical records (EMR) software; and
the help request is generated by interacting with a user-interface element integrated into a user-interface of the EMR software.

14. The apparatus of claim 11, wherein the communication session includes at least one of a phone call, videoconferencing call, or a screen sharing session.

15. The apparatus of claim 11, wherein the set of support personnel includes a first support team formed from members of an organization employing the requesting user.

16. The apparatus of claim 15, wherein the set of support personnel includes a second support team formed from members of an organization not employing the requesting user.

17. The apparatus of claim 16, wherein the selection criteria also includes a coverage time of the first support team and a coverage time of the second support team.

18. The apparatus of claim 11, wherein the help request notification is a push message.

19. The apparatus of claim 11, wherein the processor is further configured to execute the instructions to:
transmit data indicating an issue addressed during the communication session; and
transmit data indicating a solution provided to resolve the issue raised during the communication session.

20. The apparatus of claim 19, wherein the data indicating the issue addressed during the communication session and the data indicating the solution to the issue addressed during the communication session relate to issues included in future training sessions.
